(19) United States
(12) Patent Application Publication
(10) Pub. No.: US 2023/0060823 A1
(43) Pub. Date: Mar. 2, 2023

(54) THERAPY OF LAMENESS IN MAMMALS WITH PULSED RADIO FREQUENCY

(71) Applicants: Nextep B.V., Utrecht (NL); P.J.F. Anesthesiologie B.V., Roosendaal (NL)

(72) Inventors: Menno Emanuel Sluijter, UTRECHT (NL); Petrus Johannes Fransiscus de Loos, ROOSENDAAL (NL)

(21) Appl. No.: 17/463,711

(22) Filed: Sep. 1, 2021

(51) Int. Cl. A61N 1/32; A61N 1/40; A61N 1/36; A61N 1/04

(52) U.S. Cl. CPC A61N 1/326; A61N 1/40; A61N 1/36014; A61N 1/0468

(57) ABSTRACT

The present invention relates to a method of medical treatment of lameness in a mammal in need thereof, the method comprising applying pulsed radiofrequency (PRF) to the mammal so that an outcome selected from the group consisting of an improvement stance or gait, a stabilization in the decrease of a deviation from normal stance or gait, a decrease of the manifestation of pain, a stabilization in the manifestation of pain, a decrease in the mechanical restrictions causing alteration of stance or gait, a stabilization in the mechanical restrictions causing alteration of stance or gait, a decrease in neuromuscular disease, a stabilization in neuromuscular disease, and/or a decrease or stabilization of inflammation is achieved.

### FIELD OF THE INVENTION

The invention relates to the field of medicine, more particularly the field of treatment with electrical signals, more specifically pulsed radio frequency, and even more specifically to the treatment of lameness in mammals, preferably non-human, such as caninae or even-toed ungulates and odd-toed ungulates, such as equinae.

### BACKGROUND OF THE INVENTION

In the past few decades radiofrequency (RF) thermo-coagulation has been established as an accepted treatment option for several ailments. The therapeutic effect was mainly concerned with the destruction of tissue by the heat that was generated by the current. The development of a method for administrating high frequency current, pulsed radiofrequency (PRF) allowed using it to treat other pathologies and nerve structures.

With PRF, current is delivered in pulses of short duration (such as 1-50 milliseconds) separated by a silent period of about 0.1 to 0.5 second. Output current may be set not to exceed an equilibrated temperature of 42° C. to prevent cell destruction. Heat generated by the application of the current is dissipated between pulses. Nowadays PRF is recognized as treatment in humans for e.g. various forms of spinal and facial pain and peripheral neuralgias.

International application WO2008/094042A1 by one of the present inventors shows that PRF has beneficial effects on seeds and the germination of seeds. US patent publication US 2010/0198212 A1 by the present inventors shows that PRF can be used for treatment of joint pain in humans by using a needle-electrode. US patent publication US 2013/0079835 by the present inventors shows that PRF can be used for intravascular medical treatment in humans to boost the immune system and/or relieve pain, optionally together with vaccination. US patent publication US2014/0296948 A1 by one of the present inventors relates to a method of medical treatment of a mammal, preferably human suffering from cancer by applying PRF before chemotherapy or hormonal therapy either intravascularly or transcutaneously. International publication WO 2014/083203A1 by one of the present inventors relates to irregular PRF signals with a (combined) Poisson distribution of the pulse bursts, preferably in the treatment of pain, cancer, infectious diseases, COPD, depression, sequences of allostatic load, immunosuppression or otherwise caused immunodeficiency. A publication by Tavener et al. (Transcutaneous pulsed radiofrequency treatment for patients with shoulder pain booked for surgery: a double-blind, randomized controlled trial. Pain Pract 2014 feb; 14(2):101-8) discloses the transcutaneous application of PRF for shoulder pain. Each of these publications is incorporated by reference herein in its entirety.

### SUMMARY OF THE INVENTION

It has now been found by the present inventors that PRF can be used to relieve, improve, stabilize and/or solve lameness in mammals (preferably animals not including humans), in other words mammalian animals. The present invention is hence related to the treatment of mammals, preferably land-based mammals, more preferably land-based mammals weighing 2 kilograms or more, even more preferably even- and odd-toed ungulates, most preferably Equinae, such as horses or Caninae such as dogs. Equinae is a subfamily of the family Equidae and members of this subfamily are referred to as equines, the only living equines being horses, asses, and zebras of the genus Equus. Caninae is a subfamily of the family Canidae and members of this subfamily are referred to as canines, and comprising dogs, wolfs and foxes.

In a first aspect, the present invention embraces a method for medical treatment of a disorder, condition or clinical sign of lameness in mammals in need thereof, the method comprising applying pulsed radiofrequency (PRF) to the mammal so that there is: an improvement in stance or gait, a stabilization in the deviation from normal stance or gait, a decrease of the manifestation of pain, a stabilization in the manifestation of pain, a decrease in the mechanical restrictions causing alteration of stance or gait, a stabilization in the mechanical restrictions causing alteration of stance or gait, a decrease in neuromuscular disease, a stabilization in neuromuscular disease, a decrease or stabilization in (osteo)arthritis, and/or a decrease or stabilization of inflammation. In an embodiment, the invention embraces a method for medical treatment of a disorder, condition or clinical sign of lameness in mammals in need thereof, the method comprising applying pulsed radiofrequency (PRF) to the mammal so that there is a decrease in the manifestation of pain.

In other words one or more of the following effects are achieved using the PRF method of the present invention: i) the stance or gait is either improved or the deviation of normal stance or gait is stabilized/halted; ii) the decrease of the manifestation of pain or the manifestation of pain is stabilized/halted; iii) the mechanical restrictions causing alteration of stance or gait is decreased or the increase thereof is stabilized/halted; iv) neuromuscular disease caused by an abnormal stance or gait is decreased or the neuromuscular disorder or disease is stabilized/halted; v) arthritis is decreased or stabilized, vi) a decrease or stabilization of inflammation. When using the method of the present invention, typically after treatment within 2 to 14 days one or more of the above effects are observed. In case an effect is observed, treatment may be repeated. The duration of the effects of the invention may last for a period of weeks to months to years, depending on the severity of the condition and the effect obtained after the treatment. Complete elimination of lameness has been observed for periods of more than 5 to 6 months after treatment according to the present invention.

In a second aspect, the present invention embraces a method for medical treatment of overload or trauma in a mammal in need thereof comprising: i) applying at least one pair of two skin electrodes, wherein one electrode of said at least one pair of electrodes is placed on, for instance on, the head, neck, back or a leg and the other electrode of said at least one pair of electrodes is placed on the skin at the position of, for instance, the cranium, cervical vertebrae, ulna, radius, carpus, metacarpal bone, femur, tibia, fibula, calcaneus, talus, thoracic vertebrae, lumbar vertebrae, pelvis, sacral vertebrae; and ii) using a pulse generator to apply pulsed radiofrequency (PRF) over a period of between 5 and 30 minutes, having a peak current output of between 0.6 and 5.0 Ampere; to achieve an improvement in the stance or gait or stabilization or halting of the deviation of the normal stance or gait; a decrease of the manifestation of pain or a stabilization or halting thereof; a decrease of the mechanical restrictions or stabilization or halting of an increase thereof, a decrease of neuromuscular disease caused by an abnormal stance or gait or a stabilization or halting of the neuromuscular disorder or disease; a decrease or stabilization in arthritis; and/or a decrease or stabilization of inflammation.

The foregoing illustrative summary, other objectives and/or advantages of the present disclosure, and the manner in which the same are accomplished are further explained within the following detailed description.

### DETAILED DESCRIPTION

Various aspects and features are herein described. Details are set forth to provide a thorough understanding of the present disclosure. It will be apparent, however, to those having ordinary skill in the art that the disclosed PRF methods may be practiced or performed without some or all of these specific details. As another example, features disclosed as part of one embodiment can be used in another embodiment to yield a further embodiment. Sometimes, well-known aspects have not been described in detail to avoid unnecessarily obscuring the present disclosure. This detailed description is therefore not to be taken in a limiting sense, and it is intended that other embodiments are within the spirit and scope of the present disclosure.

### Disorders or Conditions

Lameness is typically defined as an abnormal stance or gait caused by e.g. a trauma, a congenital disorder, an acquired (metabolic) disorder, an infection or a disease of the nervous system of circulatory system. In addition, lameness can be noted as a change in attitude or performance. The mammal is either unwilling or unable to stand or move normally. Lameness is the most common cause of loss of use in horses. Lameness as such is not considered to be a disease but a clinical sign, a manifestation of pain, mechanical restrictions causing alteration of stance or gait. Mechanical lameness is best typified by complete upward fixation of the patella with its characteristic gait abnormality but can also be the result of fibrotic myopathy of the semitendinosus muscle or of restrictions caused by annular ligaments, adhesions, or severe fibrosis.

Alteration of stance or gait, mechanical restriction, and pain are several of the hallmarks of overload or trauma. The present inventors have found PRF to have an effect in correcting these abnormal responses and have found that it therefore can be used for treatment of overload or trauma. Stress, strain or injury can cause lameness in any mammal, even with no obvious trauma of physical defects. When lameness occurs in a mammal such as a horse, a veterinarian should be consulted. A thorough examination and subsequent successful treatment can prevent further damage to the mammal such as a horse itself and safeguard the financial investment made.

Lameness is a complicated condition, with many possible causes, and hence the treatments proposed in literature are diverse. There are several methods of treatment currently used for lameness, depending on the cause thereof. Treatment with a proper combination of systemic nonsteroidal anti-inflammatory drugs (NSAIDs) and intra-articular steroids or local analgesics is traditionally used, but mainly suitable for lameness caused by pain and not for mechanical lameness. Viscosupplementation and chondroprotectants can also be used to treat the disease and inhibit further progression of degenerative changes to the cartilage surface. Diagnostic nerve and joint blocks are also extensively described in medical literature. These analgesic techniques are both used as diagnostic and as treatment.

Since not all causes of lameness produce a characteristic gait abnormality, diagnosing remains challenging. The location of lameness can be identified by working systematically temporarily deadening sensation to specific segments of the limb, one region at a time, until the lameness disappears. This procedure isolates the area of pain causing the lameness. When lameness is observed in e.g. a leg, the origin of the problem may be in the back and/or neck. Overall, there is a need for improved methods of treating lameness in mammals.

According to the American Association of Equine Practitioners (AAEP) there is a grading scheme or score for the assessment of lameness in horses.

0: Lameness not perceptible under any circumstances.

1: Lameness is difficult to observe and is not consistently apparent, regardless of circumstances (e.g. under saddle, circling, inclines, hard surface, etc.).

2: Lameness is difficult to observe at a walk or when trotting in a straight line but consistently apparent under certain circumstances (e.g. weight-carrying, circling, inclines, hard surface, etc.).

3: Lameness is consistently observable at a trot under all circumstances.

4: Lameness is obvious at a walk.

5: Lameness produces minimal weight bearing in motion and/or at rest or a complete inability to move.

The present invention provides in an aspect, a treatment for lameness. One or more of the following effects are achieved: an improvement in stance or gait, a stabilization in the deviation from normal stance or gait, a decrease of the manifestation of pain, a stabilization in the manifestation of pain, a decrease in the mechanical restrictions causing alteration of stance or gait, a stabilization in the mechanical restrictions causing alteration of stance or gait, a decrease in neuromuscular disease, a stabilization in neuromuscular disease, a decrease or stabilization in arthritis, and/or a decrease or stabilization of inflammation.

Examples of lameness caused by overload or trauma which can be treated with the present method are the following: fibrotic myopathy, neuromuscular disorder or disease, pain-related lameness, monotonous repetitive stresses on bones, tendons, ligaments, and joints, e.g. in performance horses, e.g. upon which horses work, extremely athletic activities, direct, or indirect trauma, biomechanical laminatis, inflammation, secondary soreness in another area of the same limb, lameness in the contralateral forelimb or hindlimb from overuse due to compensation.

An example of lameness caused by a neuromuscular disorder which can be treated with the present method is arthritis (e.g., of the coffin joint), suspensory syndrome, navicular disease, bone edema and other inflammation related diseases. Examples of pain-related lameness is weight bearing (supporting leg) or non-weight bearing (swinging leg) lameness.

### Method of Application of PRF

According to the present invention, PRF may be applied locally or regionally to the affected area of the mammal body. This can be done transcutaneously or invasively.

In transcutaneous application, preferably self-adhesive, skin electrodes are attached to the skin. The purpose of transcutaneous application is to generate electromagnetic fields in a tissue compartment. The peak current that is required to achieve that is calculated first. Next, skin electrodes are attached to the skin. It may be envisaged that these skin electrodes have a surface ranging from 10 cm² to 220 cm², such as having a surface ranging from 25 cm² to 120 cm². The size of the skin electrodes to be used depends on the peak current that has been calculated. Each cm² of the skin electrodes can transport a maximum of 25 mA of peak current, assuming a duty load of 10-20 milliseconds/s. The size and type of an electrode applied can vary from one or more of the other electrodes applied. In local application the electrodes are for example placed on the head, neck, back, or leg area of the mammal to be treated, preferably near or on the neck, back, or leg, including shoulder and knee.

In invasive application one or more electrodes are used that enter into the mammal body. In such an embodiment, the method further comprises of inserting a needle or other invasive device into the body, e.g. close to a nerve in the head, neck, back or leg. In case of invasive application of the PRF, this may be by interstitial or intravitreal application of a needle or other device that can apply the pulsed radiofrequency. The size and type of an electrode applied may vary from one or more of the other electrodes applied. An example of such an electrode is a needle-electrode or other invasive device. Veterinarians might use that option, since they are familiar with techniques of injection. Such injection techniques can be combined with a PRF treatment by connecting a suitable needle to a PRF generator.

US 2010/0198212 A1 of the present inventors and incorporated by reference herein in its entirety shows the use of a specific electrode for invasive application of PRF, e.g. by a completely insulated outer needle electrode with a sharp uninsulated tip, a removable stylet, and one or more conductive, blunt ended, optionally flexible electrodes. This publication is incorporated by reference herein in its entirety.

In some preferred embodiments, PRF is applied systemically. This can for example be done transcutaneously, by attaching, preferably self-adhesive, skin electrodes to the skin. It may be envisaged that these skin electrodes have a surface ranging from 10 cm² to 220 cm². In systemic application the electrodes are placed on a body part that need not be close to the area causing the structural or functional disorder of the motor system, overload or trauma, e.g., on the legs or torso. In systemic application PRF affects a change to the behaviour of all immune cells in the mammal body.

In some preferred embodiments, PRF is applied transcutaneously after positioning at least one pair of two skin electrodes on the mammal to be treated. Since PRF is a current, a closed circuit or loop is required for the method to work. For this, one or more pairs of electrodes are used. Each pair consists of two electrodes, one electrode which functions as an active electrode sending the current and one electrode which functions as a referral electrode receiving the current. It should be noted that even two of the same type of electrodes may be used which can each carry out both functions. Each of the electrodes is applied on an opposite side of the area to be treated so that the current passes through the area to be treated. For example, when a knee is to be treated, one electrode of said pair is attached to one side of the knee and the other is attached to the other side of the knee (e.g., left and right or front and back), they may be positioned at the same height or one is above the joint to be treated whereas the other is above the joint to be treated.

In some preferred embodiments wherein PRF is applied locally and transcutaneously, one electrode of each pair of electrodes is placed on the head, neck, back or leg. In case only one pair of electrodes is used, one (the active) electrode is placed on the head, neck, back or leg, being the point of entry of the current into the mammal to be treated. The other remaining electrode (functioning as the referral electrode) may for example be placed on another part of the head, neck, back or leg of the mammal, such as on the cranium, cervical vertebrae, ulna, radius, carpus, metacarpal bone, femur, tibia, fibula, calcaneus, talus, thoracic vertebrae, lumbar vertebrae, pelvis, sacral vertebrae. In order to obtain the best effect and to be efficient it is important that the current travels through the complete affected area. In some preferred embodiments, the electrodes are placed on or in an affected limb of the subject animal. Suitable areas for placement include, but are not limited to, the hock, knee or elbow joint of the animal as well as the pelvis, coffin, gaskin, cannon, thigh, stifle, fetlock and pastern. In some preferred embodiments, the electrodes are placed on or in an affected part of the body of the subject animal. Suitable areas for placement include, but are not limited to cervical, thoracic, lumbar and sacral vertebrae.[0026] It may be advantageous to have a second pair of electrodes, for example to treat two affected areas at the same time, for instance in case of secondary sourness or lameness in contralateral limb. In that case a first electrode (functioning as active electrode) of a first pair may be placed over one affected area, and a second electrode (functioning as receiving electrode) of the first pair may be placed near the first electrode of the first pair (for instance on the other side of the joint) and a first electrode (functioning as active electrode) of a second pair may be placed over another affected area, and a second electrode (functioning as receiving electrode) may be placed near the first electrode of the second pair (for instance on the other side of another joint). Also in this case the same type of electrodes may be used which can carry out both active and receiving functions. The two affected area's may for example be the same joints on two legs of the animal.

In a particularly preferred embodiment, said mammal to be treated is a horse.

### Pulsed Radiofrequency

Pulsed radiofrequency is the technique whereby radio frequency (RF) oscillations are gated at a rate of pulses (cycles) per second (one cycle per second is known as a hertz (Hz)). In other words, pulsed radiofrequency is an electrical signal consisting of current pulses in a radiofrequency range, for example the range of 80 KHz to 10 MHz.

PRF is a clinically proven method to alleviate pain in cases where pain sensation is due to the presence of a nociceptive focus (such as in case of pain caused by pinching a nerve by a slipped disc of the spinal column, facial pain, trauma, etc.). PRF works through applying an electrical AC current to the vicinity of the nociceptive focus. The small magnetic component of the current then causes a recombination of radical pairs, thus (partially) reverting the overproduction of oxygen radicals that is the hallmark of oxidative stress (Brasil L J, Marroni N, Schemitt E, Colares J. Effects of Pulsed Radiofrequency on a Standard Model of Muscle Injury in Rats, Anesth Pain Med. Online ahead of Print; 10(1):e97372. doi: 10.5812/aapm.97372)

In some preferred embodiments, the PRF is applied over a period of between 0.5 and 60 minutes, or between 5 and 30 minutes, preferably between 10 and 20 minutes. In a specific embodiment, PRF is applied during 15 minutes.

In some preferred embodiments, the PRF pulses are delivered with a frequency of between 1 Hertz and 20 Hertz, preferably between 2 and 10 Hertz.

In some preferred embodiments, the PRF pulses are delivered with a pulse duration of between 1 millisecond and 100 milliseconds, such as between 1 and 20 milliseconds. Pulses are separated by a rest phase (also called silent period), that depends on the pulse duration and the frequency. This rest phase depends om the pulse frequency and on the pulse width (pulse duration) that have been selected. During this rest phase the voltage may be either completely turned down to 0 V or it may be significantly reduced.

In some preferred embodiments, the PRF pulses are delivered with an average duty load not exceeding 18 millisecond per second, such as when using irregular PRF. In an embodiment, the PRF pulses are delivered with an average duty load not exceeding 15 millisecond per second, such as when using regular PRF. The duty load can be calculated by multiplying the (average) pulse duration with the number of pulses per second (=pulse frequency).

PRF may be applied using regular pulses or irregular pulses. More information regarding these pulses can be found in the patent publications of the present inventor(s) disclosed above that are incorporated by reference herein. With regular pulses is meant a fixed duration for each pulse and a fixed pulse frequency. With irregular pulses is meant a varying pulse duration and a varying pulse frequency.

In some preferred embodiments, the PRF comprises regular pulses each with a single frequency and with a single pulse duration. An example thereof is a pulse duration of 5 milliseconds at a frequency of 3 Hz, leading to a duty load of 15 milliseconds per second.

In some preferred embodiments, the PRF comprises irregular pulses with an average pulse duration of between 1 and 10 milliseconds, such as 2.89 milliseconds, and an average frequency of between 1 and 20 Hz, such as 5.11 Hz. An irregular signal according to the present invention—in one embodiment—is defined as a PRF signal wherein the pulses are fired according to a Poisson distribution or a combination of Poisson distributions. See also WO 2014/083203A1 as discussed above.

In some preferred embodiments, the PRF are delivered by an electrode with an impedance of less than 1000Ω Impedance in tissue is small; this maximum value is coming from transition from electrode to skin and depends significantly on the type/manufacturer of the electrodes used.

In some preferred embodiments, the PRF is an electrical signal having an peak current output of between 0.2 and 5.0 Amperes, such as between 0.6 and 5.0 Amperes, e.g. between 1.0 and 2.0 Amperes. It should be noted that the peak current output differs from the mean current output in case of irregular pulses and also differs from transcutaneous and invasive application and from local/regional to systemic application. When using transcutaneous application it is the purpose to initiate electric fields in the magnitude that cells use for intercellular communication, the so-called physiological range (50-250 V/m) throughout the tissue compartment that is treated. At this low level cell damage due to electric fields is not possible. It is only when the electric fields are in the so-called electroporation range (10000-13500 V/m) or higher that cell damage becomes an issue. Since RF currents have a tendency to spread the current has to be calculated based on the amount of spreading that is allowed by the anatomy. It is then preferred to have a current density of 75-90 A/m².

The instruments for transcutaneous application of PRF to a patient generally comprise two electrodes, connected to a PRF current source. The two electrodes, both attached to the skin of the mammal to be treated establish an electrical circuit. The procedure to apply transcutaneous PRF is easy to perform and does not need special skills. A pair of skin electrodes (preferably self-adhesive) are applied to the skin of the mammal to be treated. The “Spring2”, a pulsed radiofrequency generator and its skin electrodes are developed and sold by Springlife Medical (www.springlife.com).

The PRF treatment is painless (except for the initial insertion of the needle electrode) and no serious and lasting adverse reactions have thus far been observed. In otherwise healthy subjects no apparent changes take place.

Other variations of the disclosed embodiments can be understood and effected by those of ordinary skill in the art in practicing the present invention by studying the drawings, the disclosure, and the appended claims. In the claims, the word “comprising” does not exclude other elements or steps, and the indefinite article “a” or “an” does not exclude a plurality. Unless otherwise specified, numerical ranges are intended to include the endpoints.

It is within the scope of this disclosure for one or more of the terms “substantially,” “about,” “approximately,” and/or the like, to qualify each adjective and adverb of the foregoing disclosure, to provide a broad disclosure. As an example, it is believed those of ordinary skill in the art will readily understand that, in different implementations of the features of this disclosure, reasonably different engineering tolerances, precision, and/or accuracy may be applicable and suitable for obtaining the desired result. Accordingly, it is believed those of ordinary skill will readily understand usage herein of the terms such as “substantially,” “about,” “approximately,” and the like.

The use of the term “and/or” includes any and all combinations of one or more of the associated listed items. Unless otherwise noted, specific terms have been used in a generic and descriptive sense and not for purposes of limitation. The invention is further elucidated by the following non-limiting examples.

### EXAMPLES

The following examples show report of the transcutaneous (example 1 and 2) and invasive (examples 3, 4 and 5) use of PRF on the affected limb(s) of five horses with lameness. The following materials and methods were used: a “Spring2” Springlife Medical model pulsed radiofrequency generator and its skin electrodes (having a size of 6×12 cm and 8×15 cm, developed and sold by Springlife Medical (on the world wide web at springlife.com). An average pulse duration of 2.89 milliseconds and an average pulse frequency of 5.11 Hertz was used in irregular phase. The duration of treatment was 15 minutes at 1.0 Amperes, three times 15 minutes at an interval of 14 days at 1.4 Amperes, respectively. For the invasive treatment the electrodes were inserted in both front legs by interstitial application. In case of invasive electrodes, the output is given in voltage and the treatment consisted of the application of regular pulses.

### Example 1—Horse 1

Horse 1 is a 13 year old gelding (Zangersheide breed) primarily used for carriage driving sports. The horse has been lame for six weeks before first visit to the clinic, it was in a good condition and had no prior complaints. The horse was examined in a straight line in both walk and trot and also on a long line on hard and soft ground. A lameness score of 3 out of 5 was diagnosed, physical examination showed no signs of swelling.

A positive diagnostic block of the lower foot was performed. Following this block, hyaluronic acid (2 ml of 20 mg/ml) was injected as well as Kenacort (1 ml of 10 mg/ml).

A significant improvement lasted for 6 months, after which there was a relapse of lameness (score 3 out of 5). X rays showed a navicular bone with deep enlarged foramina and large osteolytic zones classified as grade 4. Neurectomy was considered as a last resort, however transcutaneous pulsed radiofrequency was applied first.

The horse was treated with transcutaneous pulsed radiofrequency with an average pulse frequency of 5.11 Hz and an average pulse width of 2.89 msec (STP-mode); treatment time lasted 15 minutes at 1.0 Amperes. (PRF device: Springlife Medical, model: Spring2, CE certificate nr: CE 684095). The transcutaneous pulsed radiofrequency was administered through dedicated self-adhesive pads positioned on opposite sides of the affected limb. No local or systemic side effects of the treatment were noted.

The horse was given a rest from physical training for 3 weeks (only in the field), after which training was slowly increased. The horse now works 3 times a week for 2-3 hours in front of a carriage at a follow up of 5 months without any signs of lameness.

### Example 2—Horse 2

Horse 2 is a 15 year old gelding, Koninklijk Warmbloed Paardenstamboek Nederland (Dutch warmblood) (KWPN breed) used a dressage horse. Lameness started 18 months before the pulsed radiofrequency treatment. After the initial observation of lameness in walk and trot on a long line, X-rays and ultrasound showed a slight thickening of the tendon and slightly decreased echogenicity. After an initial full recovery following a diagnostic block (3 ml of mepivacaine 20 ml/mg) of the proximal attachment of the suspensory ligament on the left hind leg. Further therapy followed by injection of the ligament Kenacort (1 ml of 10 mg/ml). Also, a 5 daily dose of 20 ml of Meloxicam (15 mg/ml) was given for 14 days with subsequent laser type IV application of the suspensory ligament. After six months, the improvement was slight and the horse was put on a complete rest.

The horse was then again diagnosed with lameness of the left hind leg (2 out of 5) with a further extension of rest for another 6 months. Kenacort (1 ml of 10 mg/ml) was locally injected to no avail. A clearly visible thickening of the mid and proximal region of the suspensory ligament was noticed, possibly bone edema. The only option seemed to be neurectomy.

Horse 2 was instead treated with transcutaneous pulsed radiofrequency with an average pulse frequency of 5.11 Hz and an average pulse width of 2.89 msec (STP-mode); treatment time 15 minutes at 1.4 Amperes. The horse was treated 3 times with an interval of 14 days. (PRF device: Springlife Medical, model: Spring2, CE certificate nr: CE 684095). The transcutaneous pulsed radiofrequency was administered through dedicated self-adhesive pads positioned on opposite sides of the affected limb. No local or systemic side effects of the treatment were noted.

Within one week after the first PRF-treatment, improvement was clearly visible.

After the second treatment, the gait of the horse was completely sound and the intensity of training was increased. The thickening of the intermediate tendon region was reduced by 90% and was hardly visible anymore. After observation of the horse on the long line (hard and soft ground) 3 months later the horse was declared fit to compete (lameness 0 out of 5).

### Example 3—Horse 3

Horse 3 is a gelding, 10 years old, KWPN breed, jumping on Grand Prix level. Lameness made competition impossible. Veterinary examination revealed major arthritis in both front legs.

In 2008 the horse was treated invasively with PRF for 15 minutes on both front legs, after a diagnostic block with lidocaine. This was performed under ultrasound with sedation (ketamine). Two needles were placed, aligned to the nerves (n. radialis and n. ulnaris), one lateral, one medial above the hock joint on both front legs. The settings were: 5×5 ms, max temperature 40 degrees Celsius (using a NeuroTherm-generator).

A week after treatment the lameness became significantly less and after one month the horse performed in competition at CHIO Rotterdam and Spruce Meadows (Calgary).

### Example 4—Horse 4

Horse 4 is a gelding, 17 years old, Belgian Warmblood (BWP breed), eventing, at amateur level. The horse was not fit to compete for several years due to arthritis in both front legs.

The horse was treated in 2008 as described under example 3.

The horse improved and its lameness became significantly less. Although competition was not possible anymore because of his age, it was used for leisure rides in the woods for several years.

### Example 5—Horse 5

Horse 5 is a gelding, 20 years old, KWPN breed. Due to arthritis in both front legs the horse could hardly move, did not like to leave the stable and stood still during grazing.

Two weeks after treatment (same treatment as described under example 3) the horse was able to move again and started to trot and gallop.

We claim:

1. A method for medical treatment of lameness in a mammal in need thereof, the method comprising applying pulsed radiofrequency (PRF) to the mammal so that an outcome selected from the group consisting of an improvement stance or gait, a stabilization in the decrease of a deviation from normal stance or gait, a decrease of the manifestation of pain, a stabilization in the manifestation of pain, a decrease in the mechanical restrictions causing alteration of stance or gait, a stabilization in the mechanical restrictions causing alteration of stance or gait, a decrease in neuromuscular disease, a stabilization in neuromuscular disease, a decrease or stabilization in arthritis, and/or a decrease or stabilization of inflammation is achieved.

2. The method according to claim 1, wherein said lameness is caused by a disease or disorder selected from the group consisting of overload or trauma.

3. The method according to claim 1, wherein said lameness is caused by overload or trauma selected from the group consisting of fibrotic myopathy, neuromuscular disorder or disease, pain-related lameness, physical immaturity, preexisting developmental orthopedic disease, poor conformation, improper balance, improper hoof balance or shoeing, failure to adequately condition performance horses, monotonous repetitive stresses on bones, tendons, ligaments, and joints, monotonous repetitive stresses on bones, tendons, ligaments, and joints in performance horses, hard, slippery, or rocky surfaces, extremely athletic activities, direct, or indirect trauma, biomechanical laminatis, fatigue resulting in incoordination of muscles, inflammation, infection, and failure of recognition of early disease before it creates significant pain, secondary soreness in another area of the same limb, lameness in the contralateral forelimb or hindlimb from overuse due to compensation, and arthritis.

4. The method according to claim 2, wherein said pain-related lameness is selected from the group consisting of weight bearing (supporting leg) or non-weight bearing (swinging leg) lameness.

5. The method according to claim 2, wherein said physical immaturity occurs in foals selected from the group consisting of premature, dysmature or older foals trained before maturity.

6. The method according to claim 2, wherein said developmental orthopaedic disease is selected from the group consisting of osteochondrosis, flexural limb and angular limb deformities.

7. The method according to claim 1, wherein said pulsed radiofrequency is applied locally to the leg, back, neck, head or chest.

8. The method of claim 1, wherein said pulsed radiofrequency is applied transcutaneously.

9. The method according to claim 8, wherein said pulsed radiofrequency is applied after positioning at least one pair of two skin electrodes on the mammal to be treated.

10. The method according to claim 9, wherein said pulsed radiofrequency is applied transcutaneously after positioning at least one pair of two skin electrodes on the head, neck, back or leg of the mammal to be treated.

11. The method according to claim 10, wherein the electrodes are positioned on an area of the leg of the animal selected from the group consisting of the hock, knee, elbow, pelvis, coffin, gaskin, cannon, thigh, stifle, fetlock and pastern.

12. The method according to claim 10, wherein the electrodes are positioned on an area of the neck or back of the animal selected from the group consisting of cervical, thoracic, lumbar and sacral vertebrae.

13. The method of claim 1, wherein said pulsed radiofrequency is applied invasively.

14. The method according to claim 13, wherein said pulsed radiofrequency is applied after positioning at least one pair of two skin electrodes by interstitial application in the skin of the mammal to be treated.

15. The method according to claim 9, wherein said pulsed radiofrequency is applied invasively after positioning at least one pair of two skin electrodes in the skin of the head, neck, back or leg by interstitial application of the mammal to be treated.

16. The method according to claim 15, wherein the electrodes are positioned on an area of the leg of the animal selected from the group consisting of the hock, knee, elbow, pelvis, coffin, gaskin, cannon, thigh, stifle, fetlock and pastern.

17. The method according to claim 15, wherein the electrodes are positioned on an area of the neck or back of the animal selected from the group consisting of cervical, thoracic, lumbar and sacral vertebrae

18. The method according to claim 1, wherein the PRF is applied over a period of between 0.5 and 60 minutes.

19. The method according to claim 1, wherein the PRF is applied over a period of between 10 and 20 minutes.

20. The method according to claim 1, wherein the PRF is an electrical signal consisting of irregular pulses with an average pulse duration of 1-10 milliseconds and an average frequency of 2-10 Hz, or regular pulses with a pulse duration of 3-60 ms and a frequency of 2-10 Hz.

21. The method according to claim 1, wherein the PRF is an electrical signal having a peak current output of between 0.6 and 8.0 Ampere.

22. The method according to claim 1, where the mammal is a land-based mammal, preferably a land-based mammal weighing 2 kilograms or more, more preferably even- and odd-toed ungulates, and most preferably horses and dogs.

23. A method of medical treatment of fibrotic myopathy, neuromuscular disorder or disease, pain-related lameness, physical immaturity, preexisting developmental orthopedic disease, poor conformation, improper balance, improper hoof balance or shoeing, failure to adequately condition performance horses, monotonous repetitive stresses on bones, tendons, ligaments, and joints, hard, slippery, or rocky surfaces, extremely athletic activities, direct, or indirect trauma, biomechanical laminatis, fatigue resulting in incoordination of muscles, inflammation, infection, and failure of recognition of early disease before it creates significant pain, secondary soreness in another area of the same limb, lameness in the contralateral forelimb or hindlimb from overuse due to compensation, arthritis in a mammal in need thereof comprising:
i) applying at least one pair of two skin electrodes on opposite sides of the area to be treated;
ii) using a pulse generator to apply pulsed radiofrequency (PRF) over a period of between 5 and 30 minutes, having a peak current output of between 0.6 and 8.0 Ampere;
wherein said application of said PRF results in an outcome selected from the group consisting of an improvement in the stance or gait or stabilization or halting of the deviation of the normal stance or gait; a decrease of the manifestation of pain or a stabilization or halting thereof; a decrease of the mechanical restrictions or stabilization or halting of an increase thereof, a decrease of neuromuscular disease caused by an abnormal stance or gait or a stabilization or halting of the neuromuscular disorder or disease; a decrease or stabilization in arthritis; and/or a decrease or stabilization of inflammation.
